(19) United States
(12) Patent Application Publication
(10) Pub. No.: US 2021/0322156 A1
(43) Pub. Date: Oct. 21, 2021

(54) MEDICAL DEVICE BEING INSERTED INTRAVASCULARLY AND PROVIDING PROTECTION AGAINST PULMONARY HYPERTENSION RISK IN TREATMENT OF PATIENTS WITH HEART DEFECT

(71) Applicant: Mehmet GUMUSTAS, ANKARA (TR)

(72) Inventor: Mehmet GUMUSTAS, ANKARA (TR)

(21) Appl. No.: 17/273,001

(22) PCT Filed: Jul. 29, 2019
(86) PCT No.: PCT/TR2019/050631

§ 371 Date: Mar. 3, 2021

(30) Foreign Application Priority Data
Mar. 14, 2019 (TR) 2019/03805

(51) Int. Cl. A61F 2/24

(52) U.S. Cl. CPC A61F 2/2418; A61F 2210/0004; A61F 2/2433

(57) ABSTRACT

The present invention relates to a medical device (5) which is used for palliative treatment and/or treatment at every stage without developing pulmonary hypertension in patients who have increased pulmonary blood flow and pulmonary hypertension risk that are caused particularly by congenital heart diseases with left to right shunt and functional single ventricular diseases, in heart defects; also, to treat patients who have a congenital heart defect and transposition of large arteries.

### TECHNICAL FIELD

The invention disclosed relates to a medical device and application thereof which is used for the purpose of palliative treatment and/or treatment without developing pulmonary hypertension in patients of all age groups who have human heart defects, particularly congenital heart defects (for example, heart defects with left right shunt or functional single ventricle), secondary increased pulmonary blood flow and pulmonary hypertension risk, and preparing the left ventricular for systemic circulation in case of development of right ventricular failure following atrial “switch” in patients who have transposition of large arteries with a left ventricular being subjected to involution.

### BACKGROUND OF THE INVENTION

Today, main pulmonary artery is intervened by materials that provide pressure extravascularly by means of open heart surgery in patients with congenital heart defects having pulmonary hypertension risk and this approach is called as “pulmonary artery banding operation” in literature. Although post-operative pulmonary pressure is controlled, a second surgical intervention is absolutely required for every patient in this classical method. Cost, applicability and complication risk of this process are quite high.

The “pulmonary artery banding operation”, which was first applied in 1951, takes a very important place in palliation of secondary increased pulmonary blood flow for congenital heart diseases today. Being a conventional approach, the “pulmonary artery banding operation” is a surgical treatment and its results are controversial particularly in newborns and infants. Although pulmonary artery banding operation is simple in terms of surgical technique, its disadvantages are clearly known such that it is an important cause of mortality and morbidity, it definitely leads to a second surgery need, the patient is subjected to a double surgery risk and requirement of an experienced surgical team for adjusting the degree of band tightness. Besides, risks of pulmonary artery injury based on pulmonary band, occlusion of pulmonary arterial branches by band migration, secondary pulmonary valve insufficiency to pulmonary annulus dilatation, and subaortic stenosis development based on ventricular septal defect restriction in cases with aorta arising from hypoplasic ventricle are among potential complications specific to this operation. Despite all these drawbacks and disadvantages, the pulmonary artery banding operation particularly at neonatal and infancy period still continues to maintain its validity today and it is an important palliative surgery.

A patent application titled as “A Self-Expandable Medical Device Treating Defects in the Heart of a Patient” with a registration number of 2013 03951 in the state of the art is detected. When the said application is examined, the related application is an alternative using a minimally invasive approach and providing transvascular change, to a procedure which is carried out by open heart surgery of biological or mechanical valves in order to replace human heart valves. It is absolutely not possible to use the related registered medical device in indications which are same and/or similar with the disclosed invention, medically. When the medical indications, the device structures and the intracardiac hemodynamics created are examined, the inventive medical device disclosed is entirely different.

Consequently, existence of the above-stated problems and inadequacy of existing solutions made it necessary to make a development in the state of the art with respect to a medical device which is used for palliative treatment and/or treatment by protecting patients with heart defect against pulmonary hypertension risk.

### OBJECTIVE OF THE INVENTION

The present invention relates to a medical device which meets the above-mentioned requirements, will become a solution against all disadvantages and provides some additional advantages.

The main purpose of the present invention is to introduce a medical device and application thereof which is used for the purpose of palliative treatment and/or treatment without developing pulmonary hypertension in patients of all age groups who have human heart defects, particularly congenital heart defects (for example, heart defects with left right shunt or functional single ventricle), secondary increased pulmonary blood flow and pulmonary hypertension risk; and preparing the left ventricular for systemic circulation in case of development of right ventricular failure following atrial “switch” in patients who have transposition of large arteries with a left ventricular being subjected to involution.

The intracardiac hemodynamics provided by means of common conventional open heart surgery approach with the inventive medical device disclosed is created by minimum complications and cost advantages together with controllable efficacy, low risk profile without open heart surgical requirement. The disadvantages in currently known classical approach in patients with heart defect and secondary pulmonary hypertension risk are minimized with the inventive medical device which accesses to human body intravenously in a percutaneous way with a minimum interventional approach and which is inserted by means of a catheter system.

With the inventive medical device designed, a limitation is ensured in the blood flow being directed to the lungs at a “controlled” rate and it is aimed to protect both lung areas from high blood pressure and thus control pulmonary hypertension risk and reach low pulmonary vascular resistance.

Another objective of the present invention is to protect lungs at every stage without pulmonary hypertension development as of early infancy period, including neonatal period as well, and to provide conditions close to normal physiology by ensuring that ventricles are not subjected to volume and pressure load at the earliest period with the designed medical device. The invention will also minimize corrective surgery requirement by balancing left-right pressure difference in heart, reaching the conditions whereby it may help spontaneous recovery of congenital heart defects. In conditions wherein there is no need for limitation in lung blood flow and no corrective treatment is required, it can be ensured that the patient is treated by means of a second mini in urn interventional approach, access intravenously in a percutaneous way and such that it will not create any pressure difference in the main pulmonary artery, instead of subjecting the patient to a double surgery risk.

Another objective of the present invention is to provide an alternative and less risky treatment for treatment of patients who are risky for surgical treatment due to multiple disease, in unstable difficult cases for surgery.

Another objective of the present invention is to ensure that a corrective treatment can be realized in patients at older ages and/or under more suitable conditions by control of pulmonary hypertension risk and preventing them from heart failure development.

In order to achieve the above-mentioned objectives, the medical device reaches into the vessel from the skin area under local anesthesia, with a minimally invasive approach to human body, and it is inserted to the main pulmonary artery upon being advanced along vascular structures and heart by using guide wires, a catheter system that is self-expandable and/or balloon expandable and can provide blood pressure monitoring.

### BRIEF DESCRIPTION OF THE DRAWINGS

FIG. 1 is a representative view of application of the inventive medical device which is inserted to main pulmonary artery intravascularly (into vessel) in order to keep the patient from pulmonary hypertension risk, in treatment of patients with heart defect.

FIG. 2 is a view of the inventive medical device while its application to the main pulmonary artery is in the starting position.

FIG. 3 is a view of the inventive medical device while it is applied to the main pulmonary artery.

FIG. 4 is a detailed view of the inventive medical device while it is applied to the main pulmonary artery.

### REFERENCE NUMBERS

1. Lungs
2. Heart
3. Catheter systems
4. Main pulmonary artery
5. Medical device
5.1. Proximal end
5.2. Distal end

The drawings don't necessarily have to be scaled and details which are not necessary to understand the present invention may be omitted. Besides, members being at least substantially identical or having at least substantially identical functions are indicated by the same number

### DETAILED DESCRIPTION OF THE INVENTION

In this detailed description, the preferred configurations of the inventive medical device (5) are disclosed only for better understanding of the subject and such that they will not create any limiting effect.

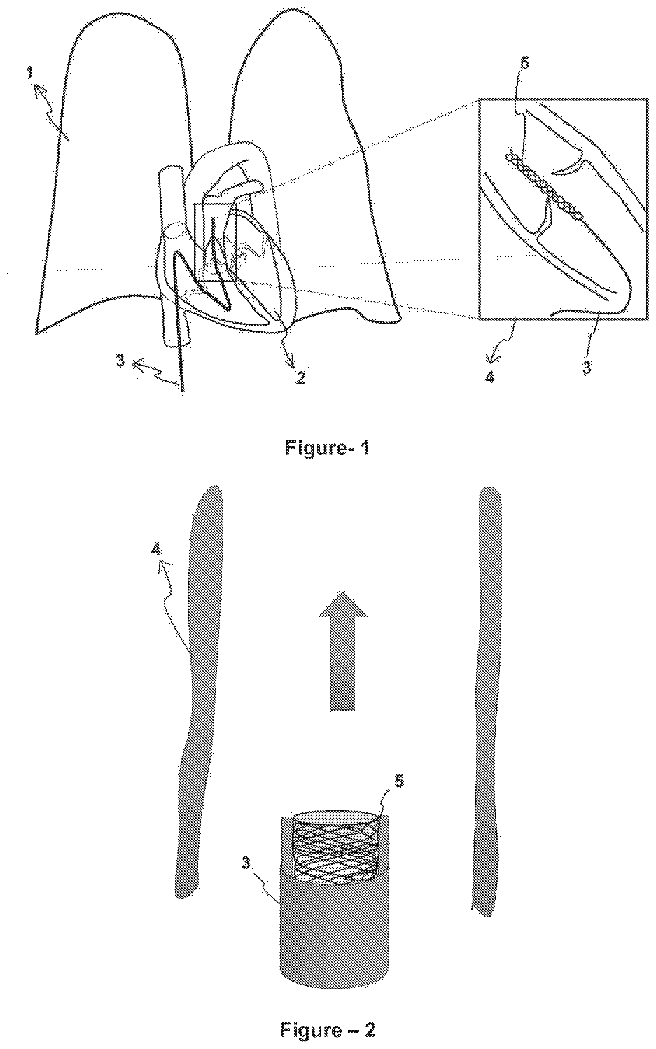

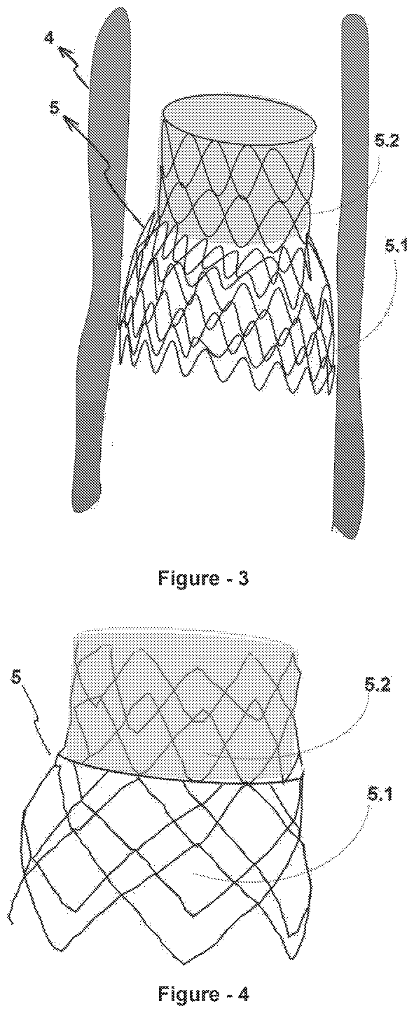

The inventive medical device (5) will be inserted to the main pulmonary artery (4) by means of a catheter system (3), that is self-expandable and/or balloon expandable, by accessing to human body with a minimum interventional approach, intravenously in a percutaneous way. The above-mentioned medical device (5) comprises a part which provides an adherence area by reaching the main pulmonary artery (4) diameter and merging with the vessel on the proximal end (5.1) close to the heart (2) whereas it comprises a cuffed area with a smaller diameter that creates resistance to blood flow on the distal end (5.2). The said medical device (5) has a stent structure consisting of a two-piece fine metallic (nitinol, etc.) foldable and flexible cage (FIG. 1).

The inventive medical device (5) reaches into the vessel from the skin area under local anesthesia, with a minimally invasive approach to human body, and it is inserted to the main pulmonary artery (4) upon being advanced along vascular structures and heart (2) by using guide wires, a catheter system (3) that is self-expandable and/or balloon expandable and can provide blood pressure monitoring. The catheter system (3) consisting of catheter and guide wires is used for transmitting the medical device (5) to the implantation site along artery or vein and for its implantation to the main pulmonary artery (4), at desired diameter (FIG. 2). The medical device (5), which is conveyed to the main pulmonary artery (4) in a folded way and with a diameter close to the catheter diameter, is carried safely along the cardiovascular pathway; it is conveyed to a second form by means of a catheter system (3) that is self-expandable and/or balloon expandable; the proximal end (5.1) diameter of the medical device (5) close to the heart is determined such that it will provide an adherence area whereas the cuffed distal end (5.2) diameter of the device is determined according to the blood pressure monitoring at the farthermost end of the catheter. A distal end (5.2) with a diameter providing a desired distal pulmonary blood pressure track is released and a “controlled” efficacy is ensured. The medical device (5) is inserted intravenously and fixed to the main pulmonary artery (4) by means of its flexible stent structure that consisting of wire-shaped elements that are linear an bendable on one another and expandable from each cell on request.

With the inventive medical device (5), blood flow directed to lungs (1) is limited at “controlled” rate and by protecting both lung (1) site from high blood pressure, pulmonary hypertension risk is controlled and low pulmonary vascular resistance is reached. In addition, by balancing left-right pressure difference in heart (2), conditions that are considered to help spontaneous recovery of congenital heart defects are reached and thus, need for corrective treatment can be reduced. In the event that there is no need for limitation in lung (1) blood flow, the medical device (5) is released into vessel upon being dilated at a rate that can reach the main pulmonary artery (4) diameter with the catheter system that is self-expandable and/or balloon expandable, such that it will not create any pressure difference in the main pulmonary artery (4) with a second minimally invasive intervention. Considering age groups and taking account of an achievable vessel diameter, dilatation degree and size of the stent is determined before implantation. Upon development of technological conditions, the device can be made of materials that are self-absorbable in body.

### Device Structure and Conditions That May be Encountered in its Implementation

Detailed anatomy of the inventive medical device (5) consists of metallic-based (nitinol, etc.) curved elements that can reach different diameters in distal end (5.2) and proximal end (5.1) parts and that are essentially interconnected. These elements are interconnected tightly via points of attachment allowing movement. Being designed in a structure that can adapt to hemodynamic changes, these elements enable to insert the medical device (5) in a balanced and firmly. However, as it is likely in implantation of every intracardiac (2) device, there may be misplacement, deregulation of the device inside the cardiovascular network, monitoring injury etc. complication in endocardial structures may be in question during insertion of the inventive medical device (5). In addition, complications and particularly vascular injury etc. which have been reported in the literature previously may be monitored in conventional access techniques provided from inguinal vessels with minimum interventional approach.

Due to the fact that the heart (2) will be subjected to dynamic forces during implantation and monitoring period of the medical device (5), based on secondary hemodynamic changes and heart cycle stages for the defect, control and management of these potential impacts on the device have importance. When these impacts are not taken into account, difficulties may develop in stabilization of the medical device (5) and complications may be monitored during and/or after implantation. Acute hemodynamic overload based on limitation in pulmonary blood flow may be another problem and if it cannot be tolerated, it may be required to the distal diameter rearrange the distal diameter with minimum interventional approach.

1. A medical device (5); characterized in that
it is intended for palliative treatment and/or treatment without developing pulmonary hypertension in patients of all age groups who have human heart defects, particularly congenital heart defects, secondary increased pulmonary blood flow and pulmonary hypertension risk;
it enables to prepare the left ventricular for systemic circulation in case of development of right ventricular failure following atrial “switch” in patients who have transposition of large arteries with a left ventricular being subjected to involution;
it reaches into the vessel from the skin area under local anesthesia, with a minimally invasive approach to human body it is inserted to the main pulmonary artery (4) upon advancing the vascular structure along the heart by using a catheter system that is self-expandable and/or balloon expandable and can provide blood pressure monitoring.

2. A medical device (5) according to claim 1; characterized in that its part which is close to the heart (2) comprises a proximal end (5.1) that provides an adherence area upon reaching the main pulmonary artery (4) diameter and merging with the vessel.

3. A medical device (5) according to claim 1; characterized in that it comprises a cuffed distal end (5.2) which creates resistance to blood flow and has a diameter less than the proximal end (5).

4. A medical device (5) according to claim 1; characterized in that it is made of a material the proximal end (5.1) and the distal end (5.2) part of which can reach diameters different from each other and which can take a metallic-based curved form that are essentially interconnected.

5. A medical device (5) according to claim 1; characterized in that it can be made of material which is self-absorbable/soluble inside human body.

6. A medical device (5) according to claim 1; characterized in that it can limit the blood flow which is directed from the heart (2) of the human wherein it is applied to his/her lungs (1), by controlling (by blood pressure monitoring) it.

7. A medical device (5) according to claim 1 and claim 6; characterized in that the medical device (5) can be released into vessel upon being dilated at a rate that can reach the main pulmonary artery (4) diameter with the catheter system that self-expandable and/or balloon expandable such that it will not create any pressure difference in the main pulmonary artery (4) with a second minimally invasive intervention, in the event that there is no need for limitation in the lung (1) blood flow of the human wherein it is applied.

8. A medical device according to claim 1; characterized in that it balances the left-light pressure difference in the heart (2) of the human wherein it is applied.

9. A medical device (5) according to claim 1; characterized in that it enables management of controllable efficacy during application by blood pressure monitoring of the said catheter system (3).
